(19) United States
(12) Patent Application Publication
(10) Pub. No.: US 2003/0233119 A1
(43) Pub. Date: Dec. 18, 2003

(54) Port deaccessor and methods of use

(76) Inventor: Margie Cheryl Tiedemann, Snellvill, GA (US)

Correspondence Address: Judy Jarecki-Black,, 467 Ware Road, Carnesville, GA, 30521, US

(21) Appl. No.: 10/174,297

(22) Filed: Jun. 18, 2002

(51) Int. Cl. A61B 17/50

(52) U.S. Cl. 606/210

(57) ABSTRACT

The present invention provides a port deaccessor comprising a forceps apparatus having a pair of arms, each having proximal and distal end portions and a middle portion spaced about halfway between the proximal and distal ends. A hinge connects the proximal portions of the arms. The distal ends provide respective distal tip portions coated with a non-stick coating such that secure placement of the distal ends of the port deaccessor around the diameter of a needle associated with an in-dwelling port or other venous line is enhanced to provide a safe and efficient means to remove the needle.

### FIELD OF THE INVENTION

The present invention relates generally to a medical device. More specifically, the present invention provides a port deaccessor comprising a forceps apparatus having a pair of arms, each having proximal and distal end portions and a middle portion spaced about halfway between the proximal and distal ends, and further including a non-slip coating on the distal ends such that placement of the distal ends of the forceps apparatus around the diameter of a port needle is secured. Use of the port deaccessor permits a caregiver to efficiently remove or deaccess the port needle from the patient's port while protecting the caregiver from an inadvertent needlestick with the contaminated needle during the removal process.

### BACKGROUND OF THE INVENTION

There are a number of implantable devices, or central venous lines, known in the conventional medical arts for gaining access to the circulatory system of a patient, when such access is required on a more than temporary basis. For example, a Peripherally Inserted Central (PIC) catheter is a line inserted by a medical practitioner into the patient's basillic or cephalic vein and then advanced into the region of the superior vena cava. The PIC, secured by means of sutures or steri-strips, is typically used for the infusion of solutions, medications, blood or blood products, and the like, as well as for obtaining blood samples from the patient without requiring additional needlesticks. Another type of central venous device, known commonly as a port, is totally implanted under the skin of a patient over a bony prominence. A port consists of two major components: a catheter, with the tip advanced into the region of the superior vena cava, and a septum, which is attached to the catheter. The septum is accessed through the patient's skin by a special port needle, one common type of which is called a Huber needle. Once the port insertion site is healed, maintenance in patients receiving on-going therapy includes changing the port needle at regular intervals.

Current methods used by medical practitioners to change the port needle usually involve placing the thumb and index finger of one hand around the diameter of the port to secure the port, while simultaneously removing or deaccessing the needle with the other hand. However, the pressure required to remove the needle from the port often causes a rebound effect on the needle. Because of this effect the practitioner is at risk of being stuck with the contaminated needle, especially in the thumb or index finger still positioned around the diameter of the port.

Pecutaneous injuries from needlesticks are a serious concern for the approximately 10 million healthcare workers in the United States alone because of the significant risk of occupational transmission of bloodborne pathogens like human immunodeficiency virus (HIV) and Hepatitis B virus, among others. Indeed, according to the Centers for Disease Control and Prevention (CDC), approximately 384,000 percutaneous injuries occur annually in U.S. hospitals, with about 236,000 of these resulting from needlesticks involving hollow-bore needles. Because the majority of the accidental needlesticks occur after the device has been used, and often when removing or deaccessing the device from the patient, there is a need for an improved method for the safe removal of needles, especially those associated with in-dwelling central venous lines. The present invention, directed to an improved forceps apparatus and method of use, provides just such a safe and convenient method for deaccessing port needles and other venous lines from a patient.

Conventional forceps are well known in the medical arts. Examples range from simple tweezers-like forceps, used to remove splinters from the skin of a patient, to more complex designs particularly adapted for a variety of medical uses. For example, U.S. Pat. No. 6,146,139 to Harrison, III discloses a forceps apparatus having particular utility in the removal of small teeth. Harrison's forceps comprise a pair of limbs having proximal, middle, and distal portions with each distal portion concavely shaped to conform to, and grip, a tooth during use.

U.S. Pat. No. 6,371,973 to Tapper discloses a forceps useful in the intrabody positioning of a medical instrument or device. Tapper's forceps comprise a first and second arm, each including a finger holding portion, a pivot portion, and a tissue holding portion, wherein the first pivot portion is attached to the second pivot portion so as to form a pivot point about which the arms co-rotate in a scissor-like motion from a grasping position to an open position and visa-versa, and where the finger-holding positions are both positioned on one side of a plane defined by the length of the first arm, the plane being perpendicular to the scissor-like motion.

Other innovations relating to medical forceps include forceps useful for vascular surgery disclosed by Saavedra in U.S. Pat. No. 6,261,308. Saavedra discloses forceps useful as vascular clamps, allowing a vascular surgeon to close torn veins while allowing the clamp to remain in place without damaging the veins.

U.S. Pat. Nos. 6,280,458, 6,245,070, and 6,293,946 also disclose improved forceps directed to a variety of medical applications. For example, U.S. Pat. No. 6,280,458 to Boche et al. discloses a surgical and holding forceps having a shaft, at least two mouth parts, and a grip that is in working engagement with a mechanism for opening and closing the mouth parts. The mechanism is acted upon by spring force such that the mouth parts are pushed in a closing direction.

U.S. Pat. No. 6,245,070 to Marquis et al., disclosing a tissue removal device used with a standard Kleppinger bipolar forceps, includes a cleaning element held on a tube fitting over the Kleppinger forceps tubes and useful for the removal of coagulated tissue from between the forceps blades.

Thorne, in U.S. Pat. No. 6,293,946, discloses non-stick forceps useful in performing electrosurgery and comprising forceps including two electrotrodes, each having a tip composed of a material having the characteristics of electrical conductivity, high thermal diffusivity, and histocompatibility.

Thus, while the foregoing body of prior art indicates it to be well known to make forceps for a variety of medical applications, the prior art described above does not teach or suggest a forceps apparatus having a pair of arms, each having proximal and distal end portions and a middle portion spaced about halfway between the proximal and distal ends, wherein the distal ends further include a non-slip coating such that secure placement of the distal ends of the forceps apparatus around the diameter of a port needle, like a Huber needle, is enhanced to aid a medical practitioner in the safe and efficient removal of the port from a patient.

### SUMMARY OF THE INVENTION

Briefly described, the present invention generally relates to a forceps apparatus and, more specifically, to a forceps apparatus having distal ends coated with a non-slip coating whereby the distal ends of the forceps are securely placing around the diameter of a medical device, such as a port needle, to permit caregivers to remove or deaccess the needle from the patient's port quickly and safely while simultaneously protecting the caregiver from an inadvertent needlestick during the removal process.

More specifically, one embodiment of a forceps apparatus made according to the present invention comprises a pair of arms, each having proximal and distal end portions and a middle portion spaced about halfway between the proximal and distal ends, wherein the distal ends further include a non-slip coating such that secure placement of the distal ends of the forceps apparatus around the diameter of a port needle is enhanced.

In another embodiment of the present invention, a forceps apparatus is contemplated that is suitable for a one-time use and is thus suitably disposed of following use of the disposable forceps apparatus to remove a port needle from the patient's port.

Yet another embodiment contemplated to be within scope of the present invention comprises a forceps apparatus having a pair of arms, each having proximal and distal end portions and a middle portion spaced about halfway between the proximal and distal ends wherein the distal ends further include a non-slip coating such that secure placement of the distal ends of the forceps apparatus around the diameter of a port needle is enhanced, and further including a first stop and a second stop, whereby each of the first and second stops is fixedly positioned on the inside concave surface of the middle portion of each arm oppositely to the other stop, such that the user can only force the arms of the forceps apparatus together to a point wherein the first stop engages the second stop.

These and other objects, features, and advantages of the present invention will become more apparent upon review of the detailed description set forth below when taken in conjunction with the accompanying figures, which are briefly described as follows.

### BRIEF DESCRIPTION OF THE FIGURES

A full and enabling disclosure of the present invention, including the best mode thereof, to one of ordinary skill in the art, is set forth more particularly in the remainder of the specification, including reference to the accompanying figures, wherein:

FIG. 1 is a perspective view of one embodiment of a port deaccessor according to the present invention.

FIG. 2 is an enlarged view of one of the tip sections of the port deaccessor of FIG. 1.

FIG. 3 is a side view of an alternate embodiment of the present invention showing the port deaccessor in an open position.

FIG. 4 illustrates the port deaccessor of FIG. 3 in a closed position.

### DETAILED DESCRIPTION OF PREFERRED EMBODIMENTS

Other objects, features and aspects of the present invention are disclosed in, or are obvious from, the following Detailed Description. It is to be understood by one of ordinary skill in the art that the present discussion is a description of exemplary embodiments only and is not intended as limiting the broader aspects of the present invention, which broader aspects are embodied in the exemplary construction. In fact, it will be apparent to those skilled in the art that various modifications and variations can be made in the present invention without departing from the scope or spirit of the invention. For instance, features illustrated or described as part of one embodiment can be used in another embodiment to yield a still further embodiment. It is intended that the present invention cover such modifications and variations as come within the scope of the appended claims and their equivalents.

The present invention provides an improved medical device useful for aiding a practitioner in the safe and efficient removal of a needle associated with an in-dwelling line or other venous port in a patient. Specifically, the port deaccessor of the present invention provides a forceps apparatus comprising a pair of arms, each having proximal and distal end portions and a middle portion spaced about halfway between the proximal and distal ends, and further including a non-slip coating on the distal ends such that secure placement of the distal ends of the forceps apparatus around the diameter of a needle like a port needle is enhanced. The use of the port deaccessor also facilitates the safe removal of a needle associated with a port or other venous device by protecting a practitioner from an inadvertent needlestick caused by a rebound effect during needle removal.

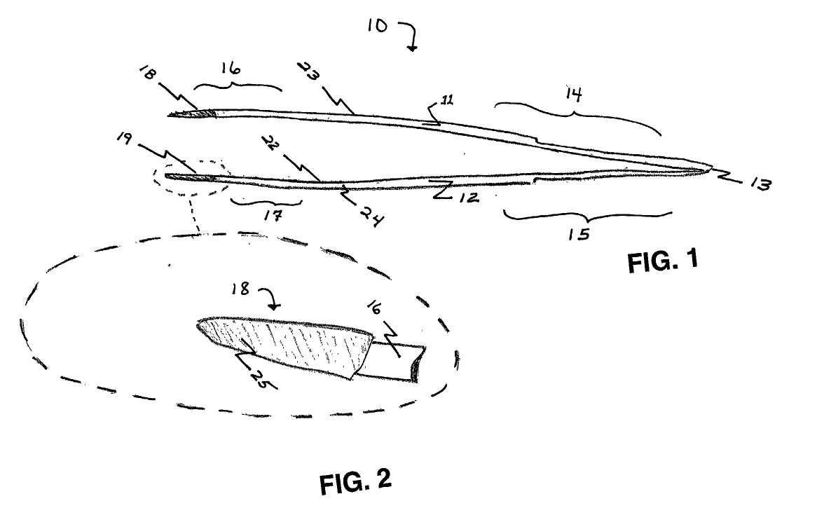

Referring now to FIGS. 1-4, port deaccessor 10 constructed in accordance with the principals of the present invention is generally illustrated. Port deaccessor 10 comprises a pair of arms 11, 12, each comprising respective proximal end portions 14, 15, respective distal end portions 16, 17, and having respective middle portions spaced about halfway between the proximal and distal ends. Proximal ends 14, 15 communicate with hinge 13. Distal end portions 16, 17 further include respective tips 18, 19 comprising a non-slip coating ensuring secure placement of the distal ends of the forceps apparatus.

Referring now to FIG. 1, illustrating one embodiment of a port deaccessor made according to the present invention, the combination of arms 11, 12 and hinge 13 can be configured such that each arm curves toward the other allowing each of tips 18, 19 to abut the other when port deaccessor 10 is in a closed position. Arms 11 thus provide concave surface 21 and convex surface 23. Similarly, arm 12 provides concave surface 22 and convex surface 24. When the practitioner forces arms 11 and 12 into a closed position, concavity 21 of arm 11 faces concavity 22 of arm 12 forcing tips 18 and 19 to converge to grip a diameter of a port needle to permit securing the needle such that removal is effectuated.

Referring now to FIG. 2, an enlarged view of one of the tip sections of port deaccessor 10 is shown, illustrating non-slip coating 25 enveloping tip 18 of distal end 16. Compositions suitable for use for non-slip coating 25 according to the present invention include, but are not limited to, rubber including synthetic rubber products, polysulfide rubbers, silicone rubbers, urethane and urethane resins, polyurethane, plastic resins including epoxy resins, polyvinylchloride, polyacrylics, polycarbonates, and the like, and mixtures thereof. One commercially available composition suitable to provide a non slip coating on the distal tips of a forceps apparatus of the present invention is LIQUID RUBBER distributed by N. J. Sarra (142 Oak Brook Commons, Clifton Park, N.Y. 12065).

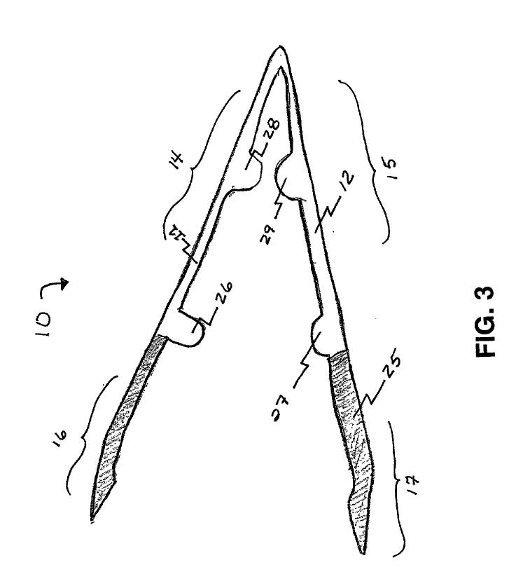

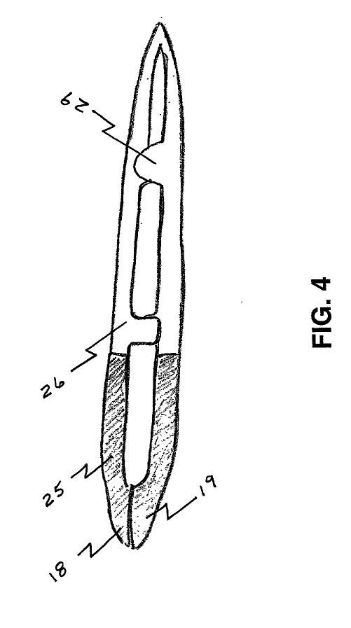

One embodiment of the present invention, illustrated in FIG. 3, contemplates port deaccessor 10 to be a forceps apparatus, measuring about 5 inches in length, and comprising arms 11, 12 and hinge 13 to be a one-piece injection molded construction. Optionally, at least one of arm 11 or 12 can include at least one stop 26 oppositely positioned to at least one slot 27 positioned on the other arm such that stop 26 engages slot 27 when a practitioner forces arms 11 and 12 into a closed position, as illustrated in FIG. 4. The present invention further contemplates port deaccessor 10 to comprise a forceps apparatus having slot 26 on distal portion 16 of arm 11 and positioned opposite to stop 27 on distal portion 17 of arm 12, while slot 29 is positioned on proximal portion of arm 12 and oppositely situated to stop 28 on proximal portion 14 of arm 11. When the practitioner forces port deaccessor 10 into a closed position, as illustrated in FIG. 4, stops 27 and 28 engage respective slots 26 and 29 allowing distal tips 18 and 19 to abut.

Suitable compositions for use in manufacturing the forceps apparatus of port deaccessor 10 include, but are not limited to, epoxy resins, plastic compositions, polyacrylics, polycarbonates, polyvinylchloride, and the like and mixtures thereof. A forceps apparatus comprising a plastic composition suitable for use in the present invention is also available commercially (EMS Medical Group Ltd., Stonehouse, Glos., England and Molded Products, Harlan Iowa, among others) and can be coated with non-stick coating by immersing distal tips 18 and 19 into a suitable rubber composition such as LIQUID RUBBER, one tip at a time, following manufacturer's directions and for approximately 10 seconds, and allowing the LIQUID RUBBER to dry. Port deaccessor 10 contemplates applying one or more coats of a non-stick coating in this fashion to provide a non-slip coating on the distal end portions of the forceps apparatus.

The port deaccessor provided according to the present invention provides an improved method and apparatus for safely removing needle associated with a port or other venous line from a patient by providing a forceps apparatus having a pair of arms, each having proximal and distal end portions and a middle portion spaced about halfway between the proximal and distal ends, and further including a non-slip coating on the distal ends such that secure placement of the distal ends of the forceps apparatus around the diameter of the needle is enhanced. The needle is then removed or deaccessed from the port by squeezing the arms of the port deaccessor together with one hand, around the diameter of the needle, while deaccessing the needle from the port with the other hand. Use of the port deaccessor to deaccess a needle from the port decreases the possibility that the practitioner will suffer an inadvertent needlestick caused by needle rebound because the needle will stick into the port deaccessor's rubber coating.

While the invention has been described with reference to specific methods and embodiments, such description is for illustrative purposes only. The words used are words of description rather than of limitation. It is to be understood that changes and variations may be made by those of ordinary skill in the art without departing from the spirit or scope of the present invention, which is set forth in the following claims.

What is claimed is:

1. A port deaccessor comprising:
a) a pair of curved limbs, each limb extending along longitudinal lines and having proximal and distal end portions and a middle portion spaced about midway in between said proximal an distal end portions, and each limb having an inside concave surface and an outer convex surface, the convex surfaces being each curved to continuously engage the thumb and forefinger of a user when the user places the forceps between the thumb and forefinger;
b) a hinge that connects the proximal ends of the limbs together;
c) the distal ends of the limbs providing respective gripping tip portions that are concavely shaped to conform to and grip a port diameter during use; and
d) each distal end further comprising a non-slip coating to secure the grip around the port diameter during use.

2. The port deaccessor of claim 1 wherein the limbs are of the same length.

3. The port deaccessor of claim 1 wherein each limb is about 2.5-7 inches in length.

4. The port deaccessor of claim 1 wherein each limb is about 5 inches in length.

5. The port deaccessor of claim 1 wherein the hinge and limbs are configured as an integral unit.

6. The port deaccessor of claim 1 comprising a plastic composition selected from the group comprising polyvinylchloride; epoxy resins; polyacrylics; polycarbonates and mixtures thereof.

7. The port deaccessor of claim 1 wherein the non-slip coating comprises a coating selected from the group comprising rubber; silicone rubber; urethane; polyurethane; an epoxy resin; polyvinylchloride; polyacrylic; polycarbonate; and mixtures thereof.

8. The port deaccessor of claim 1 wherein the non-slip coating comprises a rubber coating.
